(19) United States
(12) Patent Application Publication
(10) Pub. No.: US 2011/0021641 A1
(43) Pub. Date: Jan. 27, 2011

(54) SUSPENSION FORMULATION FOR CARBON ADSORBENTS

(76) Inventors: Olaf Behrend, Ulm (DE); Iris Heep, Koeln (DE); Nikolaus Kowollik, Merzenich-Girbelsrath (DE); Dirk Mertin, Langenfeld (DE); Petra Ohage-Spitzlei, Leverkusen (DE); Bernard Schmidt, Lindlar (DE); Wolfgang Wiehl, Koeln (DE)

Correspondence Address: BAYER HEALTHCARE LLC, P.O.BOX 390, SHAWNEE MISSION, KS, 66201, US

(21) Appl. No.: 12/922,137

(22) PCT Filed: Feb. 28, 2009
(86) PCT No.: PCT/EP2009/001450

§ 371 Date: Sep. 10, 2010

(30) Foreign Application Priority Data
Mar. 13, 2008 (DE) 10 2008 014 109.7

(51) Int. Cl. A61K 31/047; B01J 20/22; A61P 1/00

(52) U.S. Cl. 514/738; 502/401

(57) ABSTRACT

The invention relates to an aqueous suspension formulation comprising a coarsely particulate carbon adsorbent which is suitable for binding and eliminating harmful substances from the gastrointestinal tract, and a water-soluble or water-dispersible structure-forming agent and a humectant.

The invention relates to an aqueous suspension formulation comprising a coarsely particulate carbon adsorbent which is suitable for binding and eliminating harmful substances from the gastrointestinal tract, and a water-soluble or water-dispersible structure-forming agent and a humectant.

The use of carbon adsorbents such as active charcoal as binder for undesirable or toxic substances in the gastrointestinal tract has been known for a long time. Recently, porous spherical carbon adsorbents which are particularly suitable for binding and eliminating nephrotoxic compounds from the gastrointestinal tract have been developed. Such spherical carbon adsorbents have been described, for example in EP-A-29 715. EP-A-1249241, EP 1500397, EP 1525886 and EP 1745992. The carbon-based adsorbent AST-120, also referred to as kremezin, is already on the market (EP-A-1249241).

According to the prior art, these active charcoals are employed in the form of dry granules. This dry form of application, however, is only moderately suitable for use in animals, in particular cats, since it does not form a homogeneous mixture with dry animal foods. The application to dry animal food, however, is an essential prerequisite for the commercial success of such a product for animals.

A liquid or pasty formulation, however, which comprises the adsorbent can be combined with any type of animal food. The most important prerequisites for such a formulation are that the adsorption properties are not unduly reduced and that the palatability of the food or feeding stuffs is not limited either by the composition and consistency nor by the volume of the dose, even in the event of the dose inadvertently being exceeded, or in the case of top-dressing.

There is therefore a need for a formulation of carbon adsorbents which bind harmful substances and which have the following properties:
high load of adsorbent possible
adsorptive capacity retained even during storage
good palatability
easy to dose, easy to employ
formulation based on water-soluble carrier which upon oral administration mixes in a problem-free manner with the aqueous media of the gastrointestinal tract and which rapidly distributes the adsorbent freely for adsorbing the toxins which are dissolved in the aqueous medium
well-tolerated even upon prolonged administration.

The problem is solved by the formulations according to the invention.

The invention relates to:

An aqueous suspension formulation comprising a coarsely particulate carbon adsorbent, a humectant and a water-soluble on water-dispersible structure-forming agent.

“Coarsely particulate” carbon adsorbents are understood as meaning in the present context those which have a diameter of at least 0.1 mm, preferably from 0.1 to 1 mm, especially preferably from 0.1 to 0.8 mm, in particular from 0.1 to 0.6 mm. These adsorbents are preferably spherical in shape. The shape of a perfect sphere is not always possible to obtain under practice conditions and “spherical” particles are therefore also understood as meaning approximately spherical particles.

The carbon adsorbents employed in accordance with the invention preferably have a specific surface area, as determined by the BET method, of 700 m²/g or more, especially preferably 800 m²/g or more.

The carbon adsorbents may have functional groups.

Firstly, they may have acidic groups which are preferably present in a total amount of from 0.30 to 1.20 meq/g, in particular 0.30 to 1.00 meq/g.

Furthermore, the adsorbents may have basic groups which are preferably present in a total amount of from 0.20 to 0.70 meq/g, in particular from 0.30 to 0.60 meq/g.

Furthermore, the adsorbents may have phenolic hydroxyl groups which are preferably present in a total amount of from 0.20 to 0.70 meq/g.

The adsorbents may also have carboxyl groups.

In the event that the adsorbents contain functional groups, they preferably have acidic and basic functional groups, and to be precise preferably from 0.30 to 1.20 meq/g, in particular from 0.30 to 1.00 meq/g acidic groups and from 0.20 to 0.70 meq/g, in particular from 0.30 to 0.60 meq/g basic groups.

In accordance with an especially preferred embodiment of the adsorbents with functional groups, the former have a total amount of from 0.30 to 1.20 meq/g, in particular from 0.30 to 1.00 meq/g acidic groups and a total amount of from 0.20 to 0.70 meq/g, in particular from 0.30 to 0.60 meq/g basic groups, with from 0.20 to 0.70 meq/g phenolic hydroxyl groups and from 0.15 meq/g or less carboxyl groups being present.

The ratio (a:b) of the total amount of acidic groups (a) to the total amount of basic groups (b) is preferably 0.40 to 2.5. The value [(b+e)−d] for the total amount of basic groups (b), the amount of phenolic hydroxyl groups (c) and the amount of carboxyl groups (d) is preferably 0.60 or more (amount in meq/g).

The carbon adsorbents preferably have a pore volume of pores with a diameter of from 20 to 15 000 nm of from 0.04 ml/g to 0.1 ml/g, preferably from 0.05 ml/g to 0.1 ml/g.

The preparation of the carbon adsorbents is described in the prior art. As regards the preparation of the spherical carbon adsorbents, reference may be made, for example, to EP-A-29 715, EP-A-1249241. EP 1500397, EP 1525886 and EP 1745992.

It is especially preferred to employ the adsorbents described in EP-A-1249241, with the preferred embodiments described in that publication also being preferred for the present invention.

The formulations according to the invention can usually contain from 10 to 80% m/V, preferably 20 to 60% m/V, especially preferably 30 to 50% m/V of carbon adsorbent. Here and hereinbelow, “% m/V” means: weight of the constituent in question in gram per 100 ml of the finished formulation.

The formulations usually contain from 1 to 95% m/V, preferably 5 to 60% m/V, especially preferably 10 to 40% m/V of water.

The humectant is usually liquid to viscous and water-soluble. Humectants which are preferably employed are di- or trihydric alcohols with 2 to 10. preferably 3 to 6 carbon atoms, for example glycerol, ethylene glycol, diethylene glycol or propylene glycol. Preferred are propylene glycol and in particular glycerol. The humectant is usually present in the formulations in an amount of from 10 to 95% m/V, preferably from 50 to 80% m/V.

The formulations according to the invention furthermore contain water-soluble or water-dispersible structure-forming agents which, as a rule, form a gel structure with flow limit in the suspension formulations. It has emerged that the addition of a structure-forming agent improves the stability, in particular the long-term stability of the formulations, which is not easy in the case of the relatively coarsely particulate adsorbent. Suitable structure-forming agents which may be mentioned, in particular, are: cellulose derivatives, such as, for example, methylcellulose, carboxymethylcellulose, sodium carboxymethylcellulose, hydroxyethylcellulose, polymeric carbohydrates, such as, for example xanthan, alginate, gum Arabic, pectins; polypeptides, such as for example, gelatine, casein, and polyvinylpyrrolidone (PVP), ethyl acrylate and methyl methacrylate copolymers or polyacrylic acid and mixtures of such components.

It is preferred to employ a mixture of microcrystalline cellulose and sodium carboxymethylcellulose, where the mixture of the two components preferably contains from 5 to 25% (m/m), especially preferably from 7 to 20% (m/m), in particular from 10 to 15% (m/m) of sodium carboxymethylcellulose.

The structure-forming agent is usually present in amounts of from 0.2 to 15% m/V, preferably from 0.5 to 10% m/V, especially preferably from 1 to 5% m/V. If a mixture of structure-forming agents is applied, the above data refer to the total amounts.

Naturally, the formulation according to the invention can contain further customary pharmaceutical constituents, such as, for example, food colorants and/or pigments such as titanium dioxide or iron oxide. Pigments, for example, are usually present in amounts of from 0.1 to 10% m/V, preferably from 0.2 to 8% m/V.

The formulation may also contain customary preservatives such as, for example, sorbic acid if appropriate in combination with ascorbic acid. Usual concentrations which are known to the skilled worker are employed, such as, for example, from 0.01 to 1% m/V.

Preferably, however, the formulations do not contain any preservatives, especially preferably when they contain more than 30% m/V, preferably more than 40% m/V, in particular more than 50% m/V humectant.

The formulations according to the invention are liquid, preferably viscous, up to pasty consistency.

The formulations according to the invention preferably have viscosities of from 1 to 100 Pa·s, especially preferably from 1 to 30 Pa·s, very especially preferably from 5 to 20 Pa·s(measured at a shear rate of 25 s⁻¹). The formulations according to the invention are preferably distinguished by structure viscosity in their flow behaviour. Preferably, they also show thixotropism. The formulations according to the invention preferably have flow limits of from 10 to 500 Pa, in particular from 30 to 200 Pa.

The formulations according to the invention can be prepared, for example, by mixing the structure-forming agent and, if appropriate, pigments and further adjuvants into a mixture of humectant and water, or by dissolving or dispersing them therein, and subsequently incorporating the carbon-containing adsorbent into the formulation base and homogeneously distributing it therein.

The formulations according to the invention are generally useful for application in humans and animals. They are preferably employed in animal keeping and animal breeding in livestock, breeding animals, zoo animals, laboratory animals, experimental animals and companion animals, in particular in mammals.

The livestock and breeding animals include mammals such as, for example, cattle, horses, sheep, pigs, goats, camels, water buffalo, donkeys, rabbits, fallow deer, reindeer, fur bearers such as, for example, mink, chinchilla, raccoon, and birds such as, for example, chickens, geese, turkeys, ducks, pigeons and ostriches. Examples of preferred livestock are cattle, sheep, pigs and chickens.

The laboratory and experimental animals include dogs, cats, rabbits and rodents such as mice, rats, guinea pigs and golden hamsters.

The companion animals include dogs, cats, horses, rabbits, rodents such as golden hamsters, guinea pigs, mice, furthermore reptiles, amphibians, and birds which are kept in domestic premises and in zoos.

The formulations according to the invention are preferably employed in companion animals, very especially preferably in dogs and in particular cats.

They may be employed both prophylactically and therapeutically.

The formulations according to the invention are usually and preferably administered orally. Rectal administration is also possible.

The formulations according to the invention are preferably administered orally together with the animal food; they can be applied both to wet and to dry food, either admixed or else on top.

In principle, they are suitable for eliminating harmful substances from the digestive tract; the harmful substances are bound to the carbon adsorbent and eliminated. Thus, they are generally suitable for use in all states of oral poisoning such as, for example, intoxication by the oral uptake of poisons or contaminated feeds. In particular, they are suitable for the prophylaxis and treatment of kidney diseases, such as, renal insufficiencies, in particular chronic renal insufficiency and liver diseases such as, for example, hepatic insufficiency, in particular chronic hepatic insufficiency.

Examples which may be mentioned are: hepatotoxic encephalitis, chronic aflatoxin poisoning, acute states of poisoning.

As a rule, the formulations according to the invention are administered in doses such that 0.05 to 4.0. preferably 0.1 to 0.4 grams of carbon adsorbent are administered per kilogram of body weight and day.

With the conventional loading with adsorbent, typical dosage volumes are from 0.5 to 10 ml. For use in cats, for example, it is possible to prepare formulations which allow a low dosage volume of approximately from 1.25 to 5 ml per day and cat, which is advantageous for the user.

Depending on specific circumstances such as disease, animal species and the like, the dosages may diverge from what has been said.

In particular in the case of the abovementioned spherical carbon adsorbents with functional groups, such as, for example, AST-120, it is current knowledge that contact with water before application is strictly to be avoided, since the adsorption capacity of the substance for certain marker substances will otherwise be reduced. This is why exclusively dry formulations of AST-120 are currently sold in vapour-proof packaging (aluminium sachets, capsules in blister packs). Unexpectedly, it has been found that the adsorption capacity for relevant kidney toxins is retained in the formulations according to the invention even upon prolonged storage, despite the water which the formulations contain. Thus, it has been found that, for example, the adsorption capacity of AST-120 in a suspension formulation according to the invention is essentially unreduced with regard to the relevant target toxins (indole, cresol, phenol) and is comparable to the dry granules. In general, the invention therefore also relates to water-containing formulations of the spherical carbon adsorbents mentioned hereinabove and in EP-A-1249241 as being preferred and especially preferred.

The formulations according to the invention have good palatability, for example, the palatability of dry cat food is not adversely affected in the dosage range 5-20 g of AST-120/kg food.

### EXAMPLES

| | Example No. | | | | | | | |
|---|---|---|---|---|---|---|---|---|
| | 1 | 2 | 3 | 4 | 5 | 6 | 7 | 8 |
| | Content in % m/V | | | | | | | |
| AST-120 | 40 | 40 | 40 | 40 | 40 | 40 | 40 | 40 |
| Glycerol | | | 50 | 50 | 55 | 60 | 80 | 90 |
| Propylene glycol | 20 | 20 | | | | | | |
| Water | 58.3 | 58.05 | 36.25 | 37.03 | 33.25 | 29.25 | 14 | 6 |
| Titanium dioxide | 5 | 5 | 5 | 5 | 5 | 5 | 5 | 5 |
| Iron oxide pigment | | 0.25 | 0.25 | 0.25 | 0.25 | 0.25 | 0.25 | 0.25 |
| Microcrystalline | | | 2.5 | 2.5 | 2.5 | 2.5 | 2.25 | 2.5 |
| cellulose/sodium | | | | | | | | |
| carboxymethylcellulose* | | | | | | | | |
| Xanthan | 0.6 | 0.6 | | | | | | |
| Sorbic acid | | | | 0.2 | | | | |
| Ascorbic acid | | | | 0.02 | | | | |

*for example Avicel CL 611, weight ratio MCC/Na-CMC 9:1

### A. Adsorption Capacity

AST-120 is only applied as dry granules according to the prior art. Suspension formulations or pastes, in particular with a high adsorbent content, have not been described to date. The table which follows contains examples of data for the adsorption capacity of AST-120 in various paste formulations in comparison with the pure dry granules:

| | | Relative | | Relative |
|---|---|---|---|---|
| Formulation | p-cresol [mg/g] | value | Indole [mg/g] | value |
| AST-120 | 374 | 100% | 354 | 100% |
| (dry granules) | | | | |
| Example No. 3 | 369 | 98.7% | 348 | 98.3% |
| Example No. 4 | 363 | 97.1% | ′354 | 100.0% |
| Example No. 5 | 367 | 98.1% | 346 | 97.7% |
| Example No. 6 | 366 | 97.9% | 346 | 97.7% |

It can be seen that the adsorption values of all test formulations for the target toxins are, as a rule, only 2-3% and not more than 5% lower than those of the dry granules.

### B. Palatability

The palatability of examples of liquid formulations of AST-120 in or on dry cat food was tested in the dosage range of from 500 to 2000 mg per daily feed ration. The animals, which were kept in individual cages, were fed once daily as required, following the manufacturer's information (65-95 g per animal with a body weight of 2.7-4.5 kg), and the food intake time and any food leftovers were determined after no more than 2 hours. The results are compiled in the table below:

| | | | | Mean food | Food |
|---|---|---|---|---|---|
| | AST-120 | | | intake | leftovers |
| Formulation | dose (mg) | Application | N | time (min) | (g) |
| Control | 0 | n.a. | 58 | 12 | 0 |
| Example No. 1 | 500 | mixed in | 23 | 16 | 0 |
| | 2000 | mixed in | 13 | 15 | 0 |
| Example No. 2 | 500 | On top | 10 | 12 | 0 |
| | 1000 | On top | 10 | 12 | 0 |
| Example No. 3 | 500 | mixed in | 12 | 12 | 0 |
| | 2000 | mixed in | 12 | 16 | 0 |
| Example No. 4 | 500 | mixed in | 12 | 11 | 0 |
| | 2000 | mixed in | 12 | 12 | 0 |
| Example No. 6 | 500 | mixed in | 12 | 10 | 0 |
| | 2000 | mixed in | 12 | 11 | 0 |

Surprisingly, all test formulations were taken up readily together with the feed. The mean food intake time was not influenced by the addition of the formulations. No leftovers remained. This is all the more unexpected since the dose of 2000 mg of AST-120 at the tested active substance concentration in the paste already corresponds to the high dosage volume of 5 ml. With such an amount of moisture, changes in the texture of the dry food must be expected. This, however, had no effect on the acceptance of the food.

1. An aqueous suspension formulation comprising a coarsely particulate carbon adsorbent, a humectant and a water-soluble or water-dispersible structure-forming agent.

2. The suspension formulation of claim 1, wherein the humectant; is a bi- or trihydric alcohol with 2 to 10 carbon atoms.

3. The suspension formulation of claim 2, containing 10-80% m/V of carbon adsorbent.

4. The suspension formulation of claim 3, containing 10-95% m/V of humectant.

5. The suspension formulation of claim 4, containing 0.5-15% m/V of structure-forming agent.

6. The suspension formulation of claim 5 wherein the carbon adsorbent is in the form of spherical particles with a diameter of from 0.1 to 1 mm.

7. (canceled)

8. (canceled)

9. A method of removing harmful substances from the digestive tract of humans or animals, the method comprising administering the suspension formulation of claim 1 to an animal in need thereof.
